(19) United States
(12) Patent Application Publication
(10) Pub. No.: US 2026/0069864 A1
(43) Pub. Date: Mar. 12, 2026

(54) UNINTENTIONAL STIMULATION MANAGEMENT

(71) Applicant: Cochlear Limited, Macquarie University, NSW (AU)

(72) Inventor: Filiep J Vanpoucke, Huldenberg (BE)

(21) Appl. No.: 19/109,355

(22) PCT Filed: Sep. 1, 2023
(86) PCT No.: PCT/IB2023/058683

§ 371 Date: Mar. 6, 2025

Related U.S. Application Data
(60) Provisional application No. 63/406,935, filed on Sep. 15, 2022.

(51) Int. Cl. A61N 1/36; A61N 1/05

(52) U.S. Cl. CPC A61N 1/36036; A61N 1/0541

(57) ABSTRACT

The present invention relates to techniques for management of unintentional stimulation. The techniques relate to management of non-auditory stimulation, such as unintentional facial nerve stimulation, including pre-operative and intra-operative techniques for preventing unintentional non-auditory stimulation, and/or post-operative techniques for diagnosing and treating unintentional non-auditory stimulation.

### BACKGROUND

### Field of the Invention

The present invention relates generally to techniques for management of unintentional stimulation, such as unintentional non-auditory stimulation.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: obtaining a pre-operative image of an inner ear of a recipient; analyzing, with a computing device, the pre-operative image to determine a risk of unintentional facial nerve stimulation associated with insertion of a stimulating assembly into the inner ear; and outputting, with the computing device, information relating to the risk of unintentional facial nerve stimulation associated with insertion of the stimulating assembly into the inner ear.

In another aspect, a method is provided. The method comprises: following at least partial insertion of a stimulating assembly into an inner ear of a recipient; performing a plurality of electrical measurements via electrodes of the stimulating assembly to obtain a plurality of electrical parameters for a position of the stimulating assembly within the inner ear; and at a computing device, analyzing the plurality of electrical parameters to identify a risk of unintentional stimulation associated with the position of the stimulating assembly within the inner ear.

In another aspect, a method is provided. The method comprises: capturing a plurality of electrical values via electrodes of a stimulating assembly implanted in an inner ear of a recipient, wherein the stimulating assembly is a component of a medical device configured to deliver electrical stimulation signals to the inner ear; determining, based on the plurality of electrical values, that the recipient has an elevated risk of unintentional facial nerve stimulation from at least one identified electrode of the stimulating assembly; and setting at least one electrode channel configuration for use by the medical device in delivering electrical stimulation to the inner ear via the stimulating assembly to remediate the elevated risk of unintentional facial nerve stimulation from the at least one identified electrode.

In another aspect, one or more non-transitory computer readable storage media are provided. The one or more non-transitory computer readable storage media comprise instructions that are executable to: obtain a plurality of electrical values captured via electrodes of a stimulating assembly implanted in an inner ear of a recipient, wherein the stimulating assembly is a component of a medical device configured to deliver electrical stimulation signals to the inner ear; determine that the recipient has an elevated risk of stimulation side effects from at least one identified electrode of the stimulating assembly based on the plurality of electrical values; and output a recommendation for setting at least one electrode channel configuration, for use by the medical device in delivering electrical stimulation to the inner ear via the stimulating assembly, to remediate the elevated risk of stimulation side effects from the at least one identified electrode.

In another aspect, an apparatus is provided. The apparatus comprises: an input device configured to obtain a plurality of electrical measurements captured via electrodes of a stimulating assembly positioned in an inner ear of a recipient; and at least one processor configured to analyze the plurality of electrical measurements and to output an indication of a risk of unintentional non-auditory stimulation associated with the position of the stimulating assembly within the inner ear.

In another aspect, a system is provided. The system comprises: a medical device comprising a plurality of electrodes configured to be inserted in a recipient; and a computing device comprising: an input device in communication with the implantable medical device and configured to receive intraoperative measurements performed via one or more of the plurality of electrodes, and one or more processors configured to use the intraoperative measurements to estimate a risk of stimulation side effects from at least one of the plurality of electrodes and to adjust operation of the medical device to remediate the risk of the stimulation side effects.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIGS. 2A and 2B are schematic diagrams illustrating the anatomy of a human cochlea;

FIG. 2C is a schematic diagram illustrating the spatial relationship of a recipient's facial nerve function and the recipient's cochlea and internal auditory canal;

FIG. 2D is a computed tomography (CT) image showing a case of a cochlear-facial dehiscence (CFD), where the normal thin wall is absent, connecting the facial nerve canal to the cochlear labyrinth;

FIG. 3 is a flow chart illustrating an example method for pre-operative surgical management to prevent facial nerve stimulation utilizing a Pre-op Algorithm [ALGO1] according to some example embodiments;

FIG. 4 is a flow chart illustrating an example method for intra-operative surgical management to prevent facial nerve stimulation utilizing an Intra-op Algorithm [ALGO2] according to some example embodiments;

FIG. 5 is a series of graphical views illustrating cochlear outflux, as estimated by a ladder network method, in connection with the method of FIG. 4, according to some example embodiments;

FIG. 6 is a flow chart illustrating an example method for post-operative audiological management to diagnose facial nerve stimulation utilizing Post-op Diagnostic Algorithm [ALGO3] according to some example embodiments;

FIG. 7 is a flow chart illustrating an example method for post-operative audiological management to diagnose and recommend treatment for facial nerve stimulation utilizing a Post-op Recommendation Algorithm [ALGO4] according to some example embodiments;

FIG. 8A is a flow chart illustrating an example method for post-operative audiological management to diagnose and automatically treat facial nerve stimulation utilizing a fully-automated Post-op Treatment Algorithm [ALGO5] according to some example embodiments;

FIG. 8B is a graph illustrating an example where each monopolar field is modified to actively sink some current through an identified electrode (e.g., EL14), where the actively sunk current percentage through the intracochlear electrode matches the passive conduction through the facial nerve canal, in connection with the method of FIG. 8A, according to some example embodiments;

FIG. 9 is a functional block diagram of an implantable stimulator system that can benefit from the technologies described herein;

FIG. 10 is a schematic diagram illustrating a vestibular stimulator system with which aspects of the techniques presented herein can be implemented; and

FIG. 11 is a schematic diagram illustrating a retinal prosthesis system with which aspects of the techniques presented herein can be implemented.

### DETAILED DESCRIPTION

Presented herein are pre-operative, intra-operative, and post-operative techniques for management of unintentional stimulation and/or management of stimulation side effects associated with delivery of electrical stimulation of a recipient. In certain aspects, the techniques relate to management of non-auditory stimulation, such as unintentional facial nerve stimulation, including pre-operative and intra-operative techniques for preventing unintentional non-auditory stimulation, and/or post-operative techniques for diagnosing and treating unintentional non-auditory stimulation.

Merely for ease of description, the techniques presented herein are primarily described with reference to a specific implantable medical device system, namely a cochlear implant system, and with reference to a specific type of unintentional non-auditory stimulation or stimulation side effect, namely unintentional facial nerve stimulation. However, it is to be appreciated that the techniques presented herein can also be partially or fully implemented by other types of implantable medical devices for management of different types of unintentional stimulation and/or stimulation side effects. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

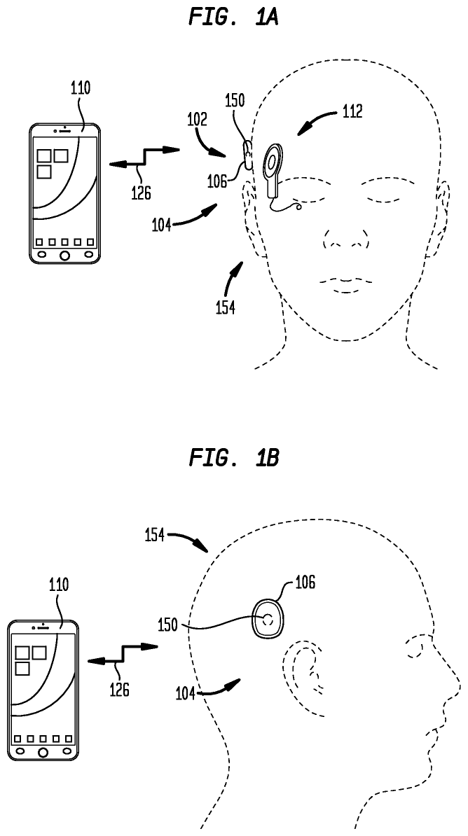

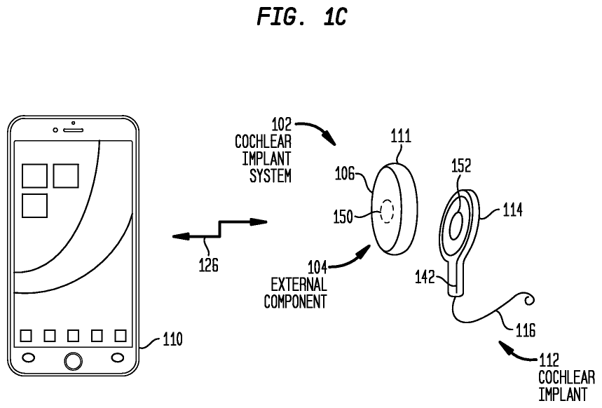

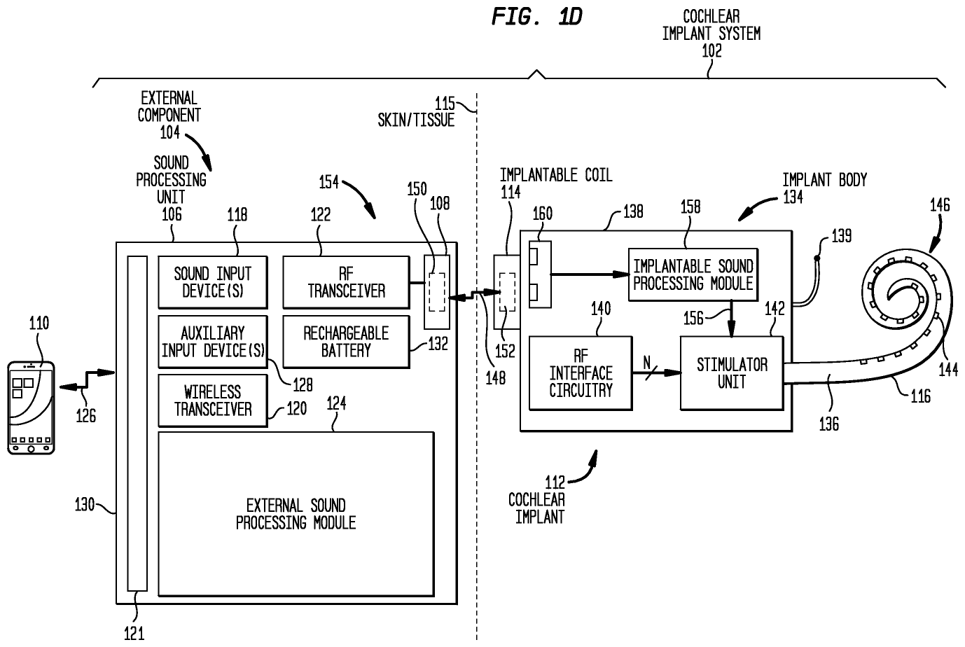

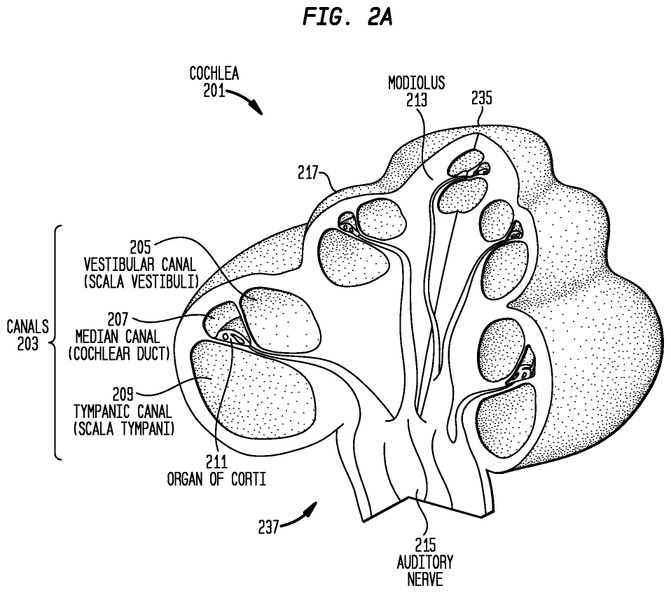

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a carrier member (e.g., a flexible silicone body) 115 with a plurality of longitudinally spaced intra-cochlear electrical stimulating contacts (electrodes) 144 disposed therein. The electrodes 144 collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

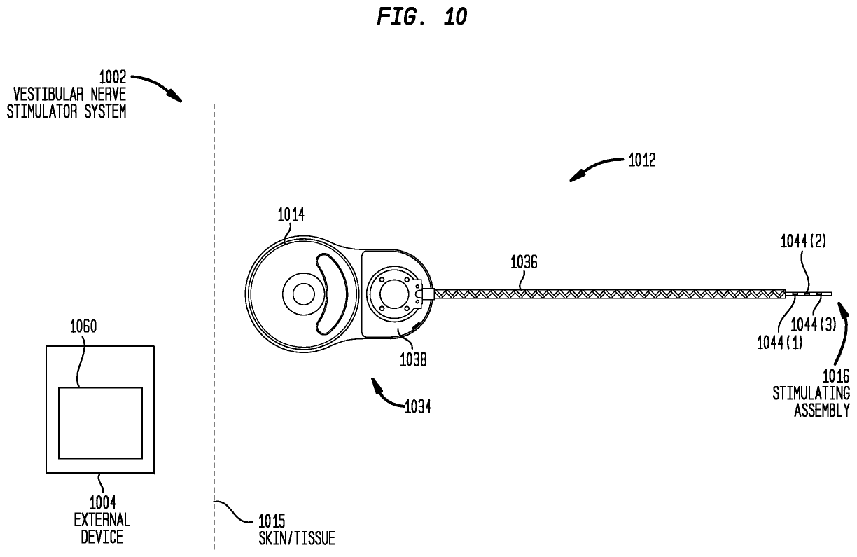

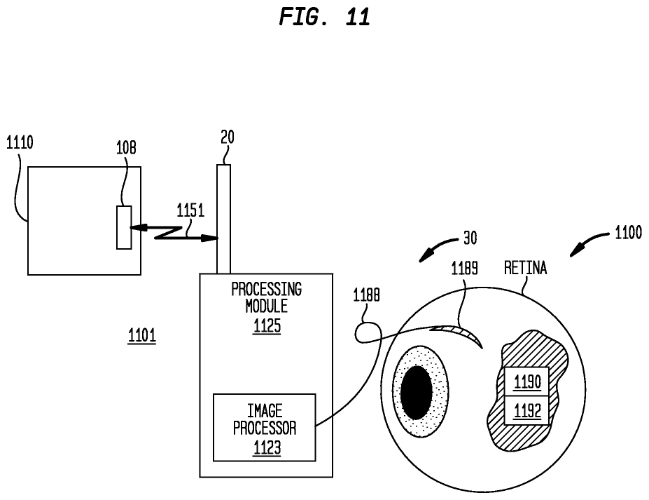

Specific example embodiments of implantable sound processing module 158 can be configured with a warped filter bank via which applications of the neural health maps are implemented in cochlear implant system 102, as described in detail with reference to FIGS. 10 and 11 below.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

Unintentional facial nerve stimulation (FNS) is one type of unintentional non-auditory stimulation (NAS) associated with, for example, stimulation of a recipient's inner ear. More specifically, unintentional facial nerve stimulation refers to unintended or unintentional activation of the facial nerve, for example as a result of intentional stimulation of other tissue/nerves of a recipient. For example, unintentional facial nerve stimulation is prevalent in cochlear implant recipients, with a reported incidence number on the order of 5-6% of recipients. In addition, the risk of facial nerve stimulation is known to be higher for certain types of stimulating assemblies (e.g., lateral straight electrodes) and in recipients with cochlear malformations.

Unintentional facial nerve stimulation causes considerable workload for medical practitioners (e.g., surgeons, audiologists, etc.) and, in many cases, medical practitioners struggle to effectively treat/remediate the problem. In general, the current audiological management techniques is, in essence, a trial-and-error approach where the medical practitioners attempt to adjust different stimulation parameters and then solicit subjective feedback from the recipient to determine if the change(s) were effective. For example, medical practitioners can: (1) identify the channels causing the unintentional facial nerve stimulation within the auditory stimulation range and lower stimulation levels, (2) change the strategy parameters, such as lowering stimulation rate and increasing pulse width, changing pulse forms (e.g., biphasic to triphasic pulses), adjusting channel configurations/polarity patterns (e.g., BP+n iso monopolar), and/or (3) deactivate the channels triggering the unintentional facial nerve stimulation.

All too often and mainly in monopolar mode, a broad set of channels contribute to the unintentional facial nerve stimulation, and therefore a wide set of channels needs to be deactivated, resulting in a compromised access to the auditory nerve (AN) and leading to poor speech outcomes. Although few studies have reported speech outcomes, it is likely that unintentional facial nerve stimulation is an important cause for poor performance with a cochlear implant.

In the unfortunate case where audiological management fails, the last option is a re-implantation, which is quite a dramatic complication. Unfortunately, re-implantation is not uncommon in these severe cases.

As can be seen from the above, medical practitioners lack techniques to effectively manage (e.g., prevent, diagnose, and/or treat) unintentional facial nerve stimulation. As such, presented herein are pre-operative, intra-operative, and post-operative techniques for management of unintentional facial nerve stimulation (e.g., pre-operative and intra-operative techniques for preventing facial nerve stimulation, and/or post-operative techniques for diagnosing and treating facial nerve stimulation).

Before explaining the techniques presented herein, it is first helpful to have a better understanding of the cochlear anatomy and relationship with/to the facial nerve.

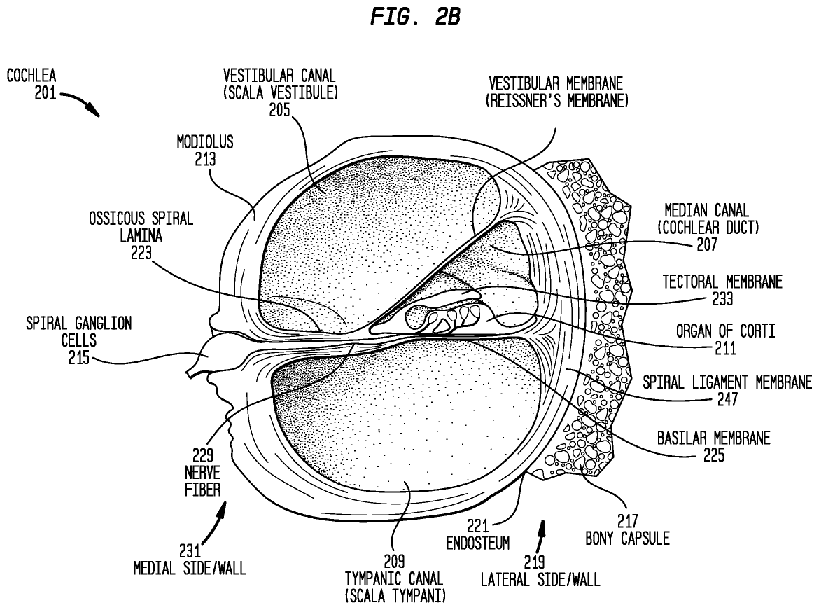

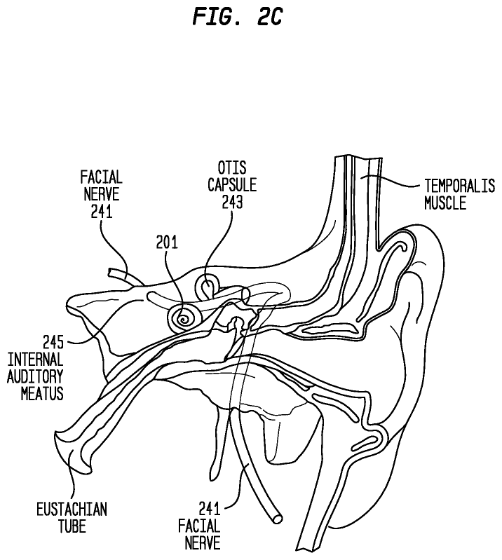

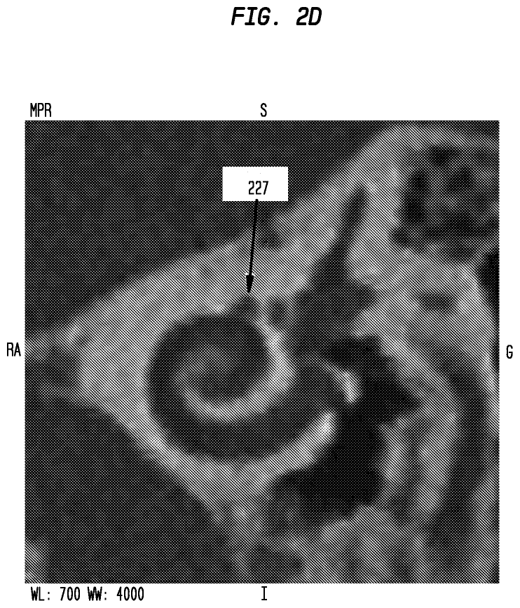

In particular, relevant aspects of an example cochlea 201 are described below with reference to FIGS. 2A-2B. More specifically, FIG. 2A is a perspective view of the cochlea 201 partially cut-away to display the canals and nerve fibers of the cochlea, while FIG. 2B is a cross-sectional view of one turn of the canals of the cochlea 201.

Referring first to FIG. 2A, cochlea 201 is a conical spiral structure comprising three parallel fluid-filled canals or ducts, collectively and generally referred to herein as canals 203. Canals 203 comprise the tympanic canal 209, also referred to as the scala tympani 209, the vestibular canal 205, also referred to as the scala vestibuli 205, and the median canal 207, also referred to as the scala media 207. Cochlea 201 spirals about modiolus 213 several times and terminates at cochlea apex 235.

Portions of cochlea 201 are encased in a bony labyrinth/capsule 217 and the endosteum 221 (e.g., a thin vascular membrane of connective tissue that lines the inner surface of the bony tissue that forms the medullary cavity of the bony labyrinth). Spiral ganglion cells 215 reside on the opposing medial side 231 (the left side as illustrated in FIG. 2B) of cochlea 201. A spiral ligament membrane 247 is located between lateral side 219 of spiral tympani 209 and bony capsule 217, and between lateral side 219 of scala media 207 and bony capsule 217. Spiral ligament 247 also typically extends around at least a portion of lateral side 219 of scala vestibuli 205.

The fluid in the tympanic canal 209 and the vestibular canal 205, referred to as perilymph, has different properties than that of the fluid which fills scala media 207 and which surrounds organ of Corti 211, referred to as endolymph. The tympanic canal 209 and the vestibular canal 205 collectively form the perilymphatic fluid space of the cochlea 201. Sound entering a recipient's auricle (not shown) causes pressure changes in cochlea 201 to travel through the fluid-filled tympanic and vestibular canals 209, 205. As noted, the organ of Corti 211 is situated on basilar membrane 225 in the scala media 207 and contains rows of 16,000-20,000 hair cells (not shown) which protrude from its surface. Above them is the tectoral membrane 233 which moves in response to pressure variations in the fluid-filled tympanic and vestibular canals 209, 205. Small relative movements of the layers of membrane 233 are sufficient to cause the hair cells in the endolymph to move thereby causing the creation of a voltage pulse or action potential which travels along the associated nerve fiber 229. Nerve fibers 229, embedded within the spiral lamina 223, connect the hair cells with the spiral ganglion cells 215 which form auditory nerve 215. Auditory nerve 215 relays the impulses to the auditory areas of the brain (not shown) for processing.

The place along basilar membrane 225 where maximum excitation of the hair cells occurs determines the perception of pitch and loudness according to the place theory. Due to this anatomical arrangement, cochlea 201 has characteristically been referred to as being “tonotopically mapped.” That is, regions of cochlea 201 toward basal region 237 are responsive to high frequency signals, while regions of cochlea 201 toward apical region 239 are responsive to low frequency signals. These tonotopical properties of cochlea 201 are exploited in a cochlear implant by delivering stimulation signals within a predetermined frequency range to a region of the cochlea that is most sensitive to that particular frequency range.

In general, the basal region 237 is the portion of the cochlea 201 located closest to the stapes (not shown in FIGS. 2A and 2B) and extends to approximately the first turn of the cochlea (i.e., the region of the cochlea 201 between the cochlea openings, including the round and oval windows, the first cochlea turn). The cochlea 201 is generally a conical spiral structure (i.e., the spiral-like shape) that terminates in the cochlear apex 235.

FIG. 2C is a schematic anatomical diagram illustrating the anatomical relationship of the facial nerve 241 relative to the cochlea 201, and shows where the facial nerve 241 passes the otic capsule 243 on its way to the internal auditory canal (internal auditory meatus) 245.

A cause of facial nerve stimulation is related to the temporal bone anatomy and relationship between the cochlea 201 and the facial nerve 241. More specifically, the facial nerve 241 is the 7^{th} cranial nerve, following the same pathway to the brainstem as the cochleovestibular nerve (8^{th} cranial nerve), running through the internal auditory canal 245. On its way into the internal auditory canal 245, the facial nerve 241 passes close to the first cochlear turn (lateral wall side) at around 270% degrees. This is called the labyrinthine section of the facial nerve 241.

At the first cochlear turn, the separation between the cochlear 201 and the facial canal is formed by a thin layer of bone and the anatomy of the facial is known to be quite variable. In some cases, the bony layer is so thin that is either just a bony shell, or is even absent. This is known as a cochlear-facial dehiscence (CFD), an example of which is illustrated in the image shown in FIG. 2D. That is, FIG. 2D is a computed tomography (CT) image illustrates a case of a recipient suffering from severe and persistent unintentional facial nerve stimulation, where no bony layer can be seen on the CT image. As shown by arrow 227, the normal thin wall is absent in FIG. 2D (which would otherwise appear as a white layer if it were present in the image), thereby connecting the facial nerve canal to the cochlear labyrinth.

A fundamental principle of tissue stimulation is that the current delivered/sourced to the tissue must be removed/sunk from the tissue (e.g., tissue has no net charge at the end of a stimulation cycle). In certain examples, cochlear implants stimulate by default in monopolar mode, in which the current flows from an intracochlear electrode (e.g., an electrode in the cochlea) through the cochlear tissues and head, then out to one or more extracochlear reference electrodes (e.g., one or more electrodes located outside of the cochlea).

Little is known about the outflow of the current in monopolar mode. However, analysis of transimpedance matrices (TIMs) indicate that the exact flow patterns are different for every recipient. Most monopolar current flows towards the base of the cochlea as the current exits the cochlea, where the cochlear aqueduct, middle ear and internal auditory canal play a role. The apex is a secondary possible current sink, but the strength of this sink varies (e.g., in some recipients, there is very little apical current drain).

The present inventors have discovered that if there is a partial or cochlear-facial dehiscence (e.g., little or no bony shell between the first turn and facial canal), then the facial canal can act as a short cut for electrical monopolar current, connecting more or less directly with a deeper section of the internal auditory canal. In general, the aggregated volume conduction through the head tissues presents a monopolar tissue impedance of +/−1000-1500 Ohm.

Now if a significant part of the current enters the facial canal, it can cause facial nerve action potentials that can be afferent and efferent, causing sensations around the eye or mouth, for example. To avoid facial nerve stimulation due to this mechanism, the key is to somehow keep the monopolar current away from the facial nerve canal. Such an approach will be more effective than adjusting stimulation parameters, such as rate, pulse wave form or pulse width. These parameters do not modify the current flow patterns, and will only have a secondary effect.

More specifically, if the labyrinthine section of the facial nerve acts as a current sink, then the nearby cochlear implant electrodes are located in the medial part of the electrode array. However, on its way through the middle ear, the facial nerve also passes relatively close to the round window. Therefore, basal electrodes can also cause unintentional facial nerve stimulation in some deviant anatomies or if the electrode is only partially inserted. In cases of otosclerosis, the current flow can also be significantly different.

The normal operation of a cochlear implant is such that the intracochlear stimulation current/charge is intended to generate a controlled loudness percept driving the auditory nerve, somewhere between threshold (T) and loud-but-comfortable (C). For the transmission of the auditory information, it is crucial that the full dynamic range (loudness) is covered. Reaching C-level requires a minimum of electrical stimulation. Facial nerve stimulation can set a maximum to the stimulation level that can be delivered on a particular electrode. Electrodes (or in general multipolar channels) will have a compromised function (less dynamic range) if unintentional facial nerve stimulation is occurring before comfortable loudness is reached. If they are ineffective even at soft levels, the better audiological approach is to disable them. This is just another condition, setting a maximum to the stimulation level (e.g., similar to compliance voltage). If this is only the case for one or two contacts, this will have no significant impact on hearing outcomes. However, once 3-4 electrodes or more need to be taken out, the electrode-neural interface is significantly compromised, leading to worse hearing outcomes, and potentially re-implantation.

Accordingly, as noted above, there is an unmet need for systems and techniques that can help prevent, diagnose and effectively treat unintentional facial nerve stimulation such that recipients no longer suffer from poor outcomes due to unintentional facial nerve stimulation. The current practices are expert approaches requiring substantial specialized experience and at risk general population of recipients are not adequately supported by any currently-existing diagnostic or treatment tools. Current unintentional facial nerve stimulation management is clinician guided, aided by the perception of the recipient. This results in an inconsistent approach that is likely to take a substantial amount of clinical time.

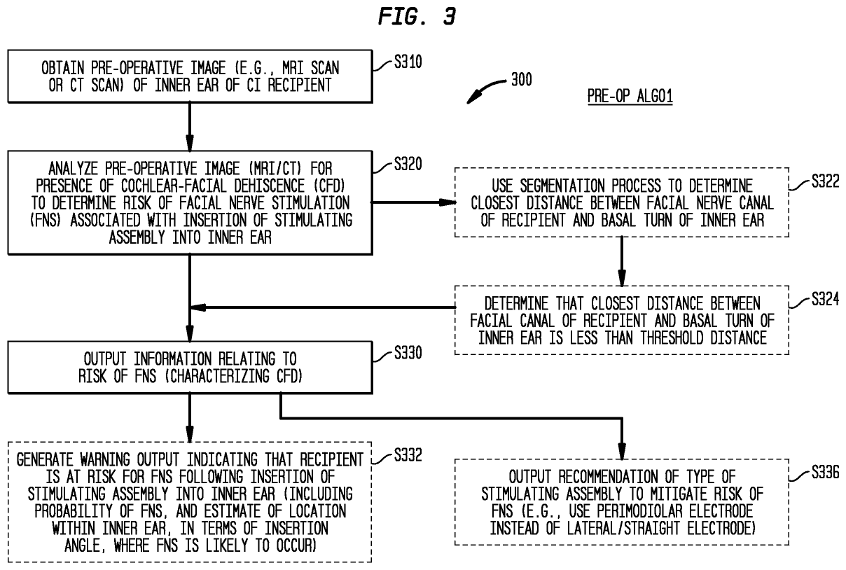

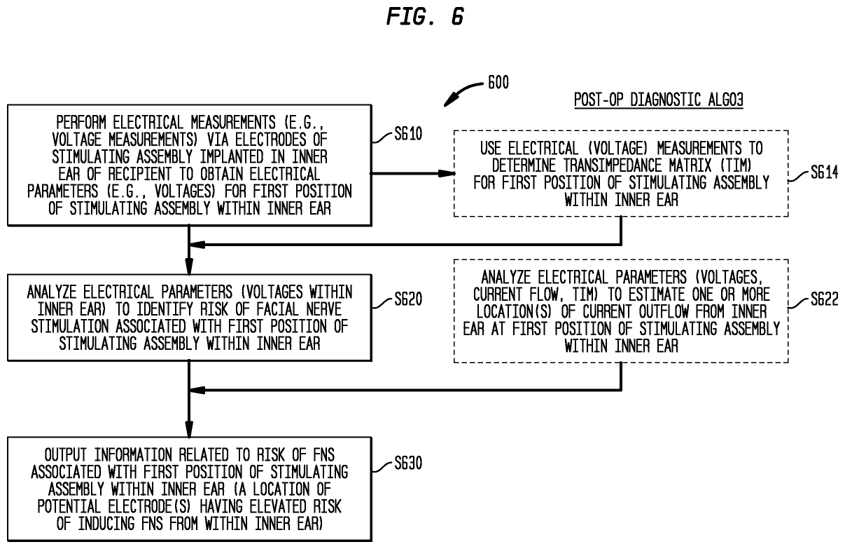

In order to address these and other needs in connection with the system, devices, and components described above with reference to FIGS. 1A-1D, presented herein are techniques for surgically and/or audiologically managing facial nerve stimulation in connection with an implantable component of an implantable medical device. In particular, presented herein are pre-operative and intra-operative techniques for preventing facial nerve stimulation, and post-operative techniques for diagnosing and treating facial nerve stimulation. The techniques presented herein provide actionable data to the surgeon or clinician, resulting in a more consistent and timely treatment of unintentional facial nerve stimulation, and can build on transimpedance measurement techniques and training with current flow models. It should be appreciated that the techniques (method steps and algorithms) described below with reference to FIGS. 3-8 can be implemented with one or more computing devices (including one or more processors and one or more memories), such as the computing devices of FIGS. 1A-1D, including but not limited to the external device 211, the external sound processing module 225 of sound processing unit 207 of external component 205, the implantable sound processing module 158 of implant body 235 of cochlear implant 213, or combinations thereof. Some of the steps and processing operations can be distributed between multiple components of the system. Next, surgical management of facial nerve stimulation will be described below with reference to FIGS. 3 and 4-5 (methods 300 and 400), and then audiological management of facial nerve stimulation will be described below with reference to FIGS. 6, 7, and 8A-8B (methods 600, 700, and 800).

### Surgical Management

Prevention of unintentional facial nerve stimulation is a favored approach, but surgeons are currently not well informed as to the risk/potential for unintentional facial nerve stimulation. In accordance with aspects presented herein, at least two tools are provided to inform the surgeon of unintentional facial nerve stimulation risk in a particular surgery.

(Pre-op ALGO1) If pre-operative computed tomography (CT) images and/or magnetic resonance imaging (MRI) images are available, the medical practitioner (e.g., surgeon, radiologist, etc.) can manually check the images for a cochlear-facial dehiscence (CFD). However, this is time consuming and cumbersome. As such, in accordance with certain embodiments presented herein, when a CT image or MRI image is available, a method according to one aspect of the present invention can use a segmentation algorithm to trace the facial nerve canal and first cochlear turn (basal turn), and determine its closest point to the basal turn (calculate or estimate the nearest geometrical distance, and possibly electrical distance by taking into account the bone density, between the facial nerve and the first cochlear turn). This distance information can then be presented to the surgeon (and/or an unintentional facial nerve stimulation probability or likelihood of future occurrence). If the distance is abnormally short, a warning can be given to the surgeon, converting the image output into a probability of unintentional facial nerve stimulation and the likely place in terms of insertion angle.

An existing surgical insertion procedure could be enriched with this feature, and, for example, the surgeon can then consider whether it would be better to choose a different electrode type (e.g., perimodiolar instead of lateral/straight) or altering the insertion technique (e.g., insertion location, position and/or angle). In some example embodiments, in addition to pre-warning for the risk of unintentional facial nerve stimulation, the pre-op algorithm (ALGO1) can also automatically suggest an electrode type (e.g., use a perimodiolar electrode) in the case of an abnormally short distance. The lateral/straight electrodes have a tendency on the outer wall of the cochlea, whereas perimodiolar electrodes spontaneously position themselves on the inner wall of the cochlea. The algorithm can be conceived as a classification algorithm (e.g., determine whether bone value is below or above a certain threshold) or it can produce a number indicating the unintentional facial nerve stimulation probability (e.g., a percentage), based on several inputs, including the electrode type. A future algorithm could potentially produce a higher unintentional facial nerve stimulation probability for the CI622 than the CI632 based on the assumed electrode position. Given enough field data, it would be possible to modulate the unintentional facial nerve stimulation probability based on the electrode location (influenced by surgical technique), for example.

As described elsewhere herein, the risk of unintentional facial nerve stimulation can depend, for example, on the mechanical attributes (i.e., type) of stimulating assembly inserted in to the cochlear. That is, different types of stimulating assemblies (e.g., perrimodiolar/midmodiolar/lateral, full ring/half ring, etc.) can have different mechanical properties that result in different trajectories and/or different implanted positions. In addition, risk of unintentional facial nerve stimulation can vary with different surgical approaches/techniques (e.g., round window insertion, cochleostomy insertion, angle of approach, insertion depth, etc.). As such, these different parameters (e.g., mechanical attributes, surgical technique, etc.) can be accounted for and the surgeon can be provided with, for example, guidance as to the selection of an appropriate stimulating assembly, surgical technique, etc.

FIG. 3 is a flow chart illustrating an example method 300 for pre-operative management to prevent facial nerve stimulation utilizing a Pre-op Algorithm [ALGO1]. Method 300 of FIG. 3 includes obtaining a pre-operative image (e.g., an MRI scan or a CT scan) of an inner ear of a recipient, as an initial step (S310). Utilizing a computing device (e.g., external device 211 of FIGS. 1A-1D, external sound processing module 225 of sound processing unit 207/external component 205, implantable sound processing module 158 of implant body 235/cochlear implant 213, or combinations thereof), method 300 includes analyzing the pre-operative image to determine a risk of facial nerve stimulation associated with insertion of a stimulating assembly into the inner ear (S320) (e.g., by analyzing for the presence of cochlear-facial dehiscence (CFD), specifically, according to an example embodiment). For example, step S320 can include the sub-steps of using an image segmentation process to determine a closest distance between the facial nerve canal of the recipient and the first cochlear turn (basal turn) of the inner ear (S322), and determining that this distance is less than a threshold distance (S324). In response to the determination of the presence of a potential risk of unintentional facial nerve stimulation occurring (distance less than threshold), method 300 further includes outputting information relating to the risk of unintentional facial nerve stimulation (and/or characterizing the detected CFD). For example, step S330 can further include the sub-step of generating a warning output indicating that the recipient is at risk for unintentional facial nerve stimulation following insertion of the stimulating assembly into the inner ear (e.g., including probability of unintentional facial nerve stimulation, and/or an estimate of a location within the inner ear, in terms of insertion angle, where unintentional facial nerve stimulation is likely to occur) (S332). Additionally or alternatively, step S330 can further include the sub-step of outputting a recommendation of a type of stimulating assembly to mitigate the risk of unintentional facial nerve stimulation (e.g., use a perimodiolar electrode, instead of a lateral/straight electrode) (S336).

The method 300 (pre-op ALGO1) is configured to determine the minimum physical distance between a recipient's facial nerve canal and the closest position to the basal turn, then convert (with use of other inputs) into surgically/clinically relevant information in the context of cochlear implantation. For example, this algorithm is implemented to find distance as a segmentation algorithm of CT images. A software feature (pre-op ALGO1) is built based on imaging and analysis of the bone layer thickness separating the cochlear labyrinth and the facial nerve canal. Method 300 (ALGO1) can indicate to the surgeon the risk of future unintentional facial nerve stimulation problems, based on anatomical analysis of the pre-op CT image (thickness measurements+thresholding). Thus, the pre-op algorithm (ALGO1) described above and in connection with method 300 of FIG. 3 can assist expert surgeons to adapt electrode type selection, surgical technique, and/or electrode position during the implantation surgery to prevent (or reduce the risk of) occurrence of unintentional facial nerve stimulation.

(Intra-op ALGO2) The second prevention technique is to diagnose and possibly adjust during surgery, before the wound is closed. If unintentional facial nerve stimulation is detected in the operating room (OR), the surgeon can intervene, such as by repositioning the electrode, for example. In certain examples, a transimpedance matrix (TIM) measurement (e.g., a 2×22 TIM measurement) can be analyzed with some advanced numerical techniques to estimate the current outflow. In some example embodiments, it can be sufficient to conduct a TIM measurement, and have a second detection/prediction algorithm run to analyze the TIM and estimate an unintentional facial nerve stimulation probability based on the TIM analysis. Therefore, a method according to another aspect of the present invention can perform a diagnostic based on a transimpedance matrix (TIM), whereby the method conducts a TIM measurement and analyzes the TIM measurement using an intelligent agent for the occurrence of a (third) current sink (e.g., a sudden change or reversal in slope), and if a third current sink occurs, the method determines its strength and extent. This number (e.g., percentage) can be shown to the surgeon, who can then decide to modify the position of the electrode or re-implant.

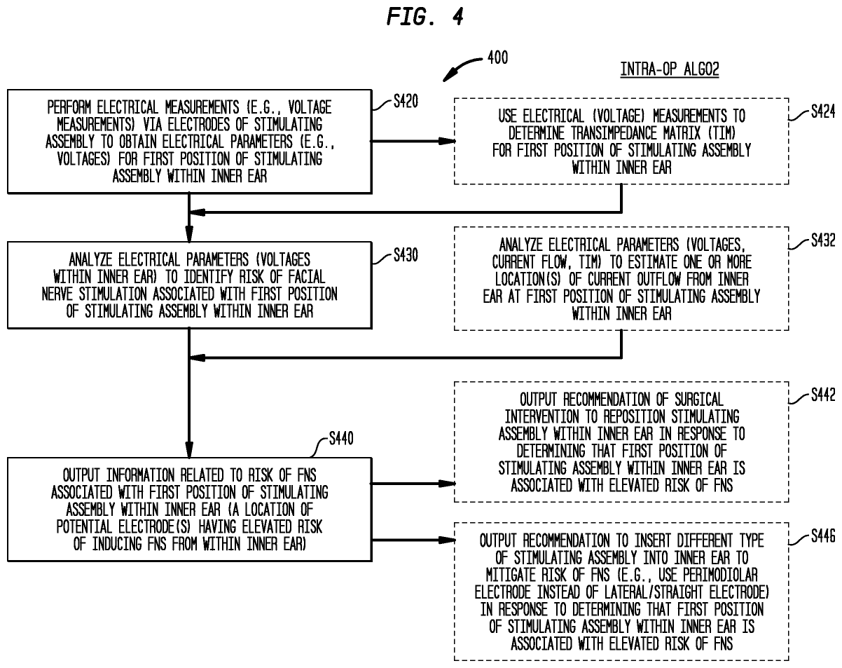

FIG. 4 is a flow chart illustrating an example method 400 for intra-operative surgical management to prevent facial nerve stimulation utilizing an intra-op Algorithm [ALGO2]. Method 400 of FIG. 4 begins at some point in time following an initial step (S410) involving the insertion (or at least a partial insertion) of a stimulating assembly into an inner ear of a recipient (not shown in FIG. 4). In some example embodiments, this time of insertion can be during the implantation surgery in an operating room. The method 400 includes performing a plurality of electrical measurements (e.g., voltage measurements) via electrodes of the stimulating assembly to obtain a plurality of electrical parameters for at least a first position of the stimulating assembly within the inner ear (S420). For example, step S420 can include the sub-step of using the plurality of electrical (voltage) measurements to determine at least one transimpedance matrix for the first position of the stimulating assembly within the inner ear (S424). Utilizing a computing device (e.g., external device 211 of FIGS. 1A-1D, external sound processing module 225 of sound processing unit 207/external component 205, implantable sound processing module 158 of implant body 235/cochlear implant 213, or combinations thereof), method 400 further includes analyzing the plurality of electrical parameters (voltages) to identify a risk of facial nerve stimulation associated with the first position of the stimulating assembly within the inner ear (S430). For example, step S430 can include the sub-step of analyzing the plurality of electrical parameters (voltages, current flow, TIM) to estimate one or more locations of current outflow from the inner ear at the first position of the stimulating assembly within the inner ear (S432). Further, method 400 can include, utilizing the computing device, outputting information related to the risk of unintentional facial nerve stimulation associated with the first position of the stimulating assembly within the inner ear (S440). For example, this information can indicate a location of one or more potential electrode(s) having an elevated risk of inducing facial nerve stimulation from within the inner ear. In addition, method 400 can include, utilizing the computing device, determining that the first position of the stimulating assembly within the inner ear is associated with an elevated risk of facial nerve stimulation (S450). In some example embodiments, step S440 can include the sub-step of outputting a recommendation of a surgical intervention to reposition the stimulating assembly within the inner ear (S442), in response to determining that the first position of the stimulating assembly within the inner ear is associated with an elevated risk of unintentional facial nerve stimulation. In some other example embodiments, step S440 can include the sub-step of outputting a recommendation to insert a different type of stimulating assembly (e.g., use perimodiolar electrode instead of lateral/straight electrode) into the inner ear to mitigate the risk of facial nerve stimulation (S446), in response to determining that the first position of the stimulating assembly within the inner ear is associated with an elevated risk of unintentional facial nerve stimulation.

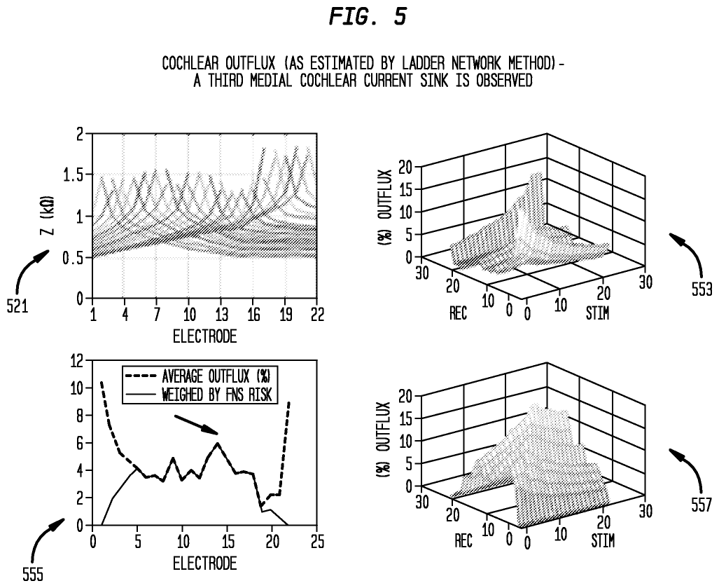

FIG. 5 is a series of graphical views illustrating cochlear outflux, as estimated by a ladder network method, in connection with method 400 of FIG. 4. There are several possible ways to analyze the TIM matrix. One method uses the lumped-element method, where a ladder model consisting of transversal and longitudinal resistors is estimated to approximate the intracochlear current flow. This is illustrated in FIG. 5 providing an example of an unintentional facial nerve stimulation case. For a person experienced in the art, the top-left TIM measurement (subplot 521 of FIG. 5) clearly demonstrates that a lot of current is exiting the cochlea near electrode 15. There is a clear change in the slopes of the “tails” at EL15. For stimulation at more basal electrodes (1-14), the more apical voltage (impedance) measurements become horizontal. No further voltage drop implies that there is no further apical-oriented current flow. All injected current has left the cochlea. Similarly, when stimulating more apical electrodes (EL16-22), there is clearly a kink at EL14. Basal slopes (voltage drops) level off past EL15, implying that either significant current has left cochlea or that the conductivity of the cochlear would have changed (where V=I×R, according to Ohm's law). The latter is highly unlikely. The top-right panel (subplot 553 of FIG. 5) shows for every stimulation electrode (monopolar use case: stimulation channel=stimulation electrode) the transversal current flow near a certain recording position. In this example, this current flow is calculated using the ladder model. Other methods could be used. Outflux is presented as a percentage of the stimulation current. For example, when stimulation at EL22, approximately 12% of the current is leaving the cochlea through the transversal resistor 22. Typically, most of the current is leaving near the base (EL1) or the apex (EL22), independent of the stimulation position.

However, and this is atypical, these slope changes are also seen near EL15, and an outflux increase is observed near EL14-15. The bottom-left panel (subplot 555 of FIG. 5) plots the outflow percentages, averaged over all stimulation positions. A peak is seen near EL14, indicating that 6% of the current leaves the cochlea near EL14, presumably because this is the place where the current can escape from the cochlea by diving into the facial canal. It can then be inferred that the occurrence of strong, significant and collocated slope changes is relatively rare and a strong indicator for unintentional facial nerve stimulation.

Assuming that reliable data sets are available, an algorithm can be designed to analyze TIM matrices for the risk of unintentional facial nerve stimulation. A third current sink does not occur often. To provide a reliable estimate of the chance of later unintentional facial nerve stimulation, a sensitive and specific unintentional facial nerve stimulation detection/prediction algorithm should be developed, using TIM or other derived features (such as the ladder network currents), as prediction features. An example generic approach is to collect a large balanced database of intra-op TIM measurements, both with and without (later) unintentional facial nerve stimulation case reports. Then a training, verification and test set can be constructed to build a reliable detector, using classic statistical classification approaches or machine learning algorithms. For further validation, clinics can use in the operating room a facial nerve monitor confirming unintentional facial nerve stimulation occurrence (e.g., by registering eye lid movement).

The bottom-right panel (subplot 557 of FIG. 5) is a first attempt towards the design of such an unintentional facial nerve stimulation prediction algorithm. The transversal currents are weighed with a function indicating the probability of unintentional facial nerve stimulation (pFNS) as a function of Itrans (transversal current flow). In the example, it is simply assumed that the first 5 apical and basal electrodes have a decreased probability of generating unintentional facial nerve stimulation (noting that this assumption could be based on real data if available, or could be estimated from field data in the future). Clearly a peak near EL14 is observed.

Such an intra-op unintentional facial nerve stimulation predictor can provide the surgeon with info in the operating room, at a time where a surgical intervention is still possible, e.g., by repositioning the electrode. Possibly, an advanced form of such an algorithm can indicate the degree of unintentional facial nerve stimulation (e.g., Is a strong current sink? What is the likely extent, e.g., will only a few electrodes generate unintentional facial nerve stimulation or not?, etc.). If a surgeon is also applying facial nerve monitoring during the operating room, it should be even possible to estimate the stimulation level causing unintentional facial nerve stimulation, and compare to this to typical C-levels in the population or recipient-specific tNRT values (in case these would contain a facial nerve contribution).

Therefore, method 400 (intra-op ALGO2) according to some example embodiments performs electrical impedance measures to locate the presence of a current sink being indicative of potential facial nerve stimulation (this approach is deployed intra-operatively during surgery in the OR), and convert to surgically/clinically relevant information in the context of cochlear implantation (e.g., implemented as a ladder network model for TIM measurements). A diagnostic tool is provided for conducting electrical measurements (e.g., identifying voltages and/or the outgoing current patterns) (ALGO2), and analyzing a transimpedance matrix (TIM) to indicate the risk of facial nerve stimulation, and/or identifying the electrodes that can cause the unintentional facial nerve stimulation. This decision algorithm (ALGO2) is smart enough (through machine learning from examples or other artificial intelligence) to predict whether a clinical problem is likely to arise, and under what conditions (e.g., which electrodes, which stimulation levels), so that the surgeon can apply this while still in the OR. This can require building a data set of problematic cases and normal cases, and identifying various features (not necessarily the ladder network analysis) that feed into a classification algorithm. Thus, the intra-op algorithm (ALGO2) described above and in connection with method 400 of FIGS. 4-5 can assist expert surgeons to adapt electrode selection, surgical technique, and/or to re-position the electrode while in the operating room during the implantation surgery to prevent (or reduce the risk of) occurrence of unintentional facial nerve stimulation.

### Audiological Management

If the surgeon has not detected unintentional facial nerve stimulation in the operating room (e.g., via Intra-op ALGO2, method 400), the unintentional facial nerve stimulation can be detected during the first fitting session with the audiologist. Again, the audiologist is currently not informed. The overall incidence data are known, but there is no diagnostic tool informing the audiologist of unintentional facial nerve stimulation risk, prior to the activation session. If during the activation mapping, the recipient mentions non-auditory-stimulation, the audiologist uses his or her own experience, essentially trying a trial-and-error process to determine which channels (or electrodes) cause unintentional facial nerve stimulation and at what stimulation levels, and whether to disable these channels/electrodes or not. The main determinant here should be whether sufficient loudness can be provoked prior to the occurrence of an annoying facial nerve percept.

(Diagnostic ALGO3) A much better situation would be if the audiologist can run (pro-actively or retro-actively) the TIM measurement and unintentional facial nerve stimulation prediction algorithm. This diagnostic tool will generate workflow efficiencies for the healthcare or medical practitioner, since the practitioner (e.g., audiologist) will receive info near which electrodes the current sink is occurring. The audiologist can then concentrate efforts on this location. It should be appreciated that post-op TIMs differ somewhat from intra-op TIMs (e.g., due to the fibrosis process). It is therefore likely that the post-op unintentional facial nerve stimulation prediction tool, described above for the intra-op case of FIG. 4, will need a different training set. As described below, the unintentional facial nerve stimulation diagnostic tool could contain an unintentional facial nerve stimulation thresholding procedure where the minimum stimulation level is determined to evoke unintentional facial nerve stimulation. Once this threshold is known, it will be possible to estimate the maximum stimulation levels on other electrodes as well (e.g., using the ladder network model).

Thus, the post-op algorithm (ALGO3) performs a diagnostic during the first fit to diagnose FNS, and the method involves the localization of the facial nerve (in terms of electrodes) and informing the audiologist of the likely location of the most sensitive channel (electrode) provoking unintentional facial nerve stimulation. This diagnostic algorithm can be guided with prior knowledge (training data set) from operating room analysis, if available (e.g., from Intra-op ALGO2 for the same recipient and/or other similar case studies). The post-op algorithm (ALGO3) described above and below in connection with FIG. 6 can help audiologists or other healthcare professionals to deal with complex cases of unintentional facial nerve stimulation sometime after the implantation surgery.

FIG. 6 is a flow chart illustrating an example method 600 for post-operative audiological management to diagnose unintentional facial nerve stimulation utilizing Post-op Diagnostic Algorithm [ALGO3]. Method 600 of FIG. 6 begins at some point in time following an initial step involving the implantation of a stimulating assembly into an inner ear of a recipient (not shown in FIG. 6). The method 600 includes performing electrical measurements (e.g., voltage measurements) via electrodes of the stimulating assembly implanted in the inner ear of the recipient to obtain electrical parameters (e.g., voltages) for a first position of the stimulating assembly within the inner ear (S610). For example, step S610 can include the sub-steps of using the electrical (voltage) measurements to determine a transimpedance matrix (TIM) for the first position of the stimulating assembly within the inner ear (S614). Utilizing a computing device (e.g., external device 211 of FIGS. 1A-1D, external sound processing module 225 of sound processing unit 207/external component 205, implantable sound processing module 158 of implant body 235/cochlear implant 213, or combinations thereof), method 600 further includes analyzing the electrical parameters (voltages) to identify a risk of unintentional facial nerve stimulation associated with the first position of the stimulating assembly within the inner ear (S620). For example, step S620 can include the sub-step of analyzing the electrical parameters (e.g., voltages, current flow, TIM) to estimate one or more locations of current outflow from the inner ear at the first position of the stimulating assembly within the inner ear (S622). Further, method 600 can include, utilizing the computing device, outputting information related to the risk of unintentional facial nerve stimulation associated with the first position of the stimulating assembly within the inner ear (S630). For example, this information can indicate a location of a potential electrode that has an elevated risk of inducing unintentional facial nerve stimulation from within the inner ear.

Thus, in some example embodiments, method 600 of FIG. 6 involves performing electrical impedance measures to locate the presence of a current sink being indicative of potential unintentional facial nerve stimulation (this approach is deployed post-operatively at the clinic), and converting to surgically/clinically relevant information in the context of cochlear implantation (e.g., implemented as a ladder network model for TIM measurements). A diagnostic tool for identifying the outgoing current patterns (ALGO3) is provided, which analyzes the transimpedance matrix to indicate the risk of unintentional facial nerve stimulation, and identifies the electrodes that can cause the unintentional facial nerve stimulation. This decision algorithm (ALGO3) is smart enough (through machine learning from examples) to predict whether a clinical problem is likely to arise, and under what conditions (e.g., which electrodes, which stimulation levels), so that the audiologist can apply this at the time of activation after the surgery has already been completed. This can also require building a data set of problematic cases and normal cases, and identifying various features (not necessarily the ladder network analysis) that feed into a classification algorithm.

If unintentional facial nerve stimulation is occurring in a recipient, a treatment technique that involves reprogramming the device can still allow the recipient to hear well. The audiologist has at least two options of recommendation and treatment tools according to the following additional aspects of the present invention, which can build on the techniques described above in connection with the diagnostic ALGO3 and method 600 of FIG. 6.

(Recommendation ALGO4) A first option is to optimize the recipient's “map” (e.g., sound processing settings) without changing the stimulation strategy. A first recommendation tool can assist the audiologist in the remapping, and can inform the medical practitioner by estimating maximum stimulation levels on each electrode or channel, instead of measuring them. If one facial nerve threshold value (e.g., on the closest electrode) is known, the TIM measurement can be used to estimate the maximum stimulation levels on other positions. The algorithm can also recommend electrode/channel deactivation. Again, the diagnostic tool can contain an unintentional facial nerve stimulation thresholding procedure where the minimum stimulation level is determined to evoke unintentional facial nerve stimulation, and once this threshold is known, it is possible to estimate the maximum stimulation levels on the other electrodes as well (e.g., using the ladder network model). Such an algorithm can also optimize secondary parameters, such as rate, pulse width and/or pulse shape. However, it should be noted that the effectiveness of parameter tuning can be somewhat limited in practice (e.g., optimizing the rate, pulse width and/or pulse shape can only help for mild cases of unintentional facial nerve stimulation in some instances).

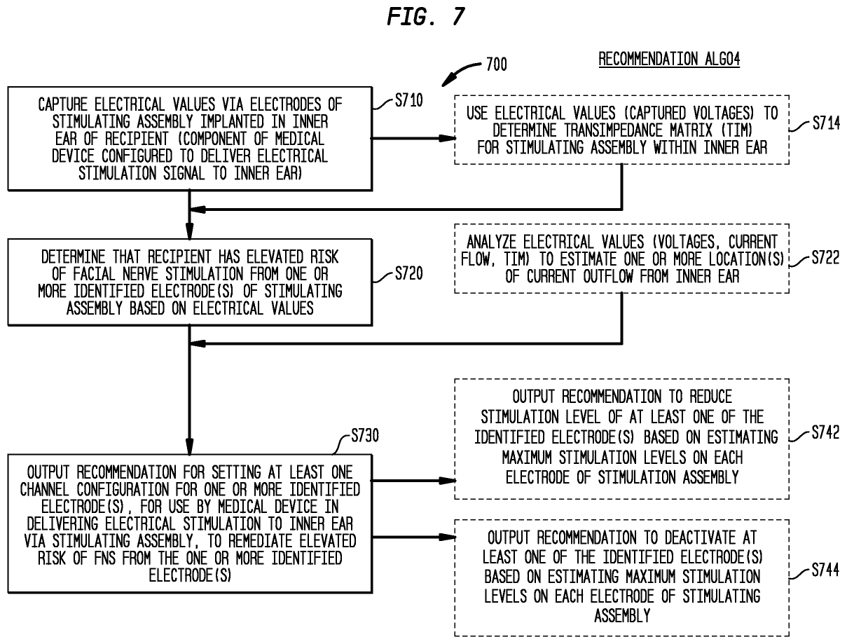

FIG. 7 is a flow chart illustrating an example method 700 for post-operative audiological management to diagnose and recommend treatment for unintentional facial nerve stimulation utilizing a Post-op Recommendation Algorithm [ALGO4]. Method 700 of FIG. 7 begins at some point in time following an initial step involving the implantation of a stimulating assembly into an inner ear of a recipient (not shown in FIG. 7). In some example embodiments, this time of insertion can be after the implantation surgery in the operating room has been completed, such as during an initial fitting with an audiologist, or some other future time. The method 700 includes capturing electrical values (e.g., voltages) via electrodes of the stimulating assembly implanted in the inner ear of the recipient (S710), where the stimulating assembly is a component of a medical device configured to deliver electrical stimulation signal to the inner ear. For example, step S710 can include the sub-step of using the electrical values (captured voltages) to determine a transimpedance matrix (TIM) for the stimulating assembly within the inner ear (S714). Utilizing a computing device (e.g., external device 211 of FIGS. 1A-1D, external sound processing module 225 of sound processing unit 207/external component 205, implantable sound processing module 158 of implant body 235/cochlear implant 213, or combinations thereof), method 700 further includes determining that the recipient has an elevated risk of unintentional facial nerve stimulation from one or more identified electrode(s) of the stimulating assembly based on the electrical values (S720). For example, step S720 can include the sub-step of analyzing the electrical values (e.g., voltages, current flow, TIM) to estimate one or more locations of current outflow from the inner ear (S722). Further, method 700 can include, utilizing the computing device, outputting a recommendation for setting at least one channel configuration for the one or more identified electrode(s), for use by the medical device in delivering electrical stimulation to the inner ear via the stimulating assembly, to remediate the elevated risk of unintentional facial nerve stimulation from the one or more identified electrode(s) (S730). For example, this information can indicate a location of a potential electrode that has an elevated risk of inducing unintentional facial nerve stimulation from within the inner ear. In addition, method 700 can include, utilizing the computing device, estimating maximum stimulation levels on each electrode of the stimulating assembly (S740), where the recommendation output at step S730 can be determined based on these estimates. In some example embodiments, step S730 can include the sub-step of outputting a recommendation to reduce the stimulation level of at least one of the one or more identified electrode(s) to a level below an upper limit to remediate the elevated risk of unintentional facial nerve stimulation (S742), based on the estimated maximum stimulation levels for each electrode of the stimulating assembly. In some other example embodiments, step S730 can include the sub-step of outputting a recommendation to deactivate at least one of the one or more identified electrode(s) to remediate the elevated risk of unintentional facial nerve stimulation (S744), based on the estimated maximum stimulation levels of each electrode of the stimulating assembly.

Thus, the post-op recommendation algorithm (ALGO4) described above and in connection with method 700 of FIG. 7 builds on the diagnostic algorithm (ALGO3), where the method involves map creation for supporting treatment. In some example embodiments, this recommendation algorithm can recommend monopolar map optimizations (e.g., to reconfigure channels to limit levels, and/or deactivate certain channels/electrodes).

(Treatment ALGO5) A second option is to reconfigure the stimulation channels, avoiding that any current is flowing along the labyrinthine section of the FN. In this example embodiment, the electronic platform will determine the intervention options and automatically implement a recommended treatment option. For some stimulating assemblies, the options are bipolar channels and common ground channels. In these approaches, all current is taken back from within the cochlea. These stimulation modes will require higher stimulation levels to excite the auditory nerve, and drain battery, but will avoid facial perception. unintentional facial nerve stimulation. An algorithm can help the audiologist to select these channels.

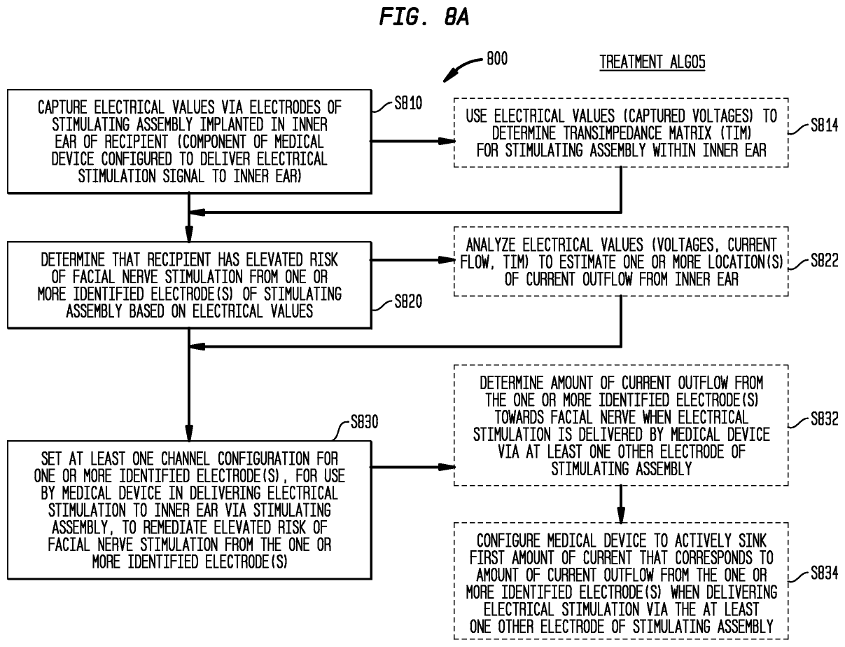

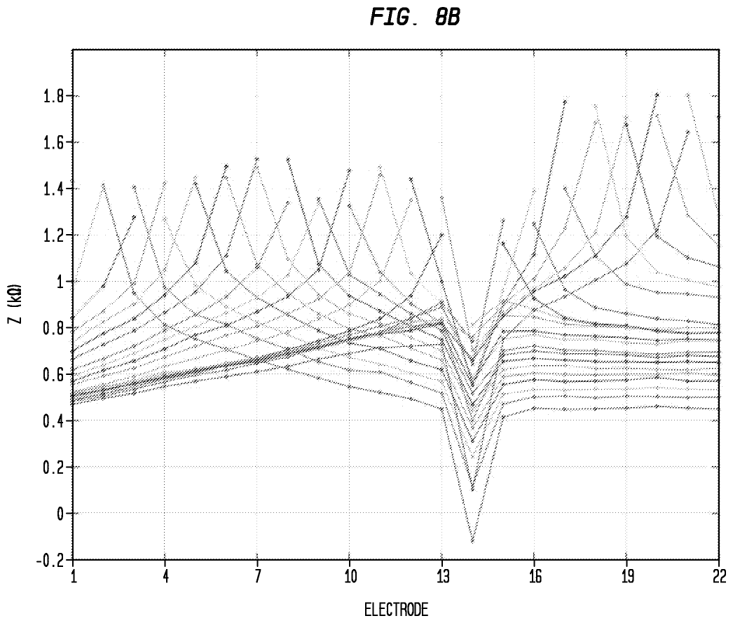

FIG. 8A is a flow chart illustrating an example method 800 for post-operative audiological management to diagnose and automatically treat unintentional facial nerve stimulation utilizing a Post-op Treatment Algorithm [ALGO5]. Method 800 of FIG. 8A begins at some point in time following an initial step involving the implantation of a stimulating assembly into an inner ear of a recipient (not shown in FIG. 8A). In some example embodiments, this time of insertion can be after the implantation surgery in the operating room has been completed, such as during an initial fitting with an audiologist, or some other future time. The method 800 includes capturing electrical values (e.g., voltages) via electrodes of the stimulating assembly implanted in the inner ear of the recipient (S810), where the stimulating assembly is a component of a medical device configured to deliver electrical stimulation signal to the inner ear. For example, step S810 can include the sub-step of using the electrical values (captured voltages) to determine a transimpedance matrix (TIM) for the stimulating assembly within the inner ear (S814). Utilizing a computing device (e.g., external device 211 of FIGS. 1A-1D, external sound processing module 225 of sound processing unit 207/external component 205, implantable sound processing module 158 of implant body 235/cochlear implant 213, or combinations thereof), method 800 further includes determining that the recipient has an elevated risk of unintentional facial nerve stimulation from one or more identified electrode(s) of the stimulating assembly based on the electrical values (S820). For example, step S820 can include the sub-step of analyzing the electrical values (e.g., voltages, current flow, TIM) to estimate one or more locations of current outflow from the inner ear (S722). Further, method 800 can include, utilizing the computing device, setting at least one channel configuration for one or more identified electrode(s), for use by the medical device in delivering electrical stimulation to the inner ear via the stimulating assembly, to remediate the elevated risk of unintentional facial nerve stimulation from the one or more identified electrode(s) (S830). For example, step S830 can further include the sub-steps of determining an amount of current outflow from the one or more identified electrode(s) towards the facial nerve when electrical stimulation is delivered by the medical device via at least one other electrode of the stimulating assembly (S832), and configuring the medical device to actively sink a first amount of current that corresponds to the amount of current outflow from the one or more identified electrode(s) when delivering electrical stimulation via the at least one other electrode of the stimulating assembly (S834).

The post-op treatment algorithm (ALGO5) described above and in connection with method 800 of FIG. 8A further builds on the diagnostic algorithm (ALGO3) and recommendation algorithm (ALGO4), where the method involves suggesting optimal changes to the spatial channel definition that would keep the (monopolar) current away from the facial nerve canal, and automatically implementing these optimal changes according to its recommendation. The new channel definitions can depend on the capabilities of the chip set, an analysis of the intracochlear electrical fields, and can suggest changes from monopolar to bipolar or all-polar (common ground) in the case of simple electrical circuits (e.g., CIC4). However, the new channel definitions can also be more advanced and personalized based on a precision analysis of the TIM measurements.

The multipolar capability of certain implants offers better options to redefine channels. Bipolar and common-ground configurations have a significant drawback that the battery life will be compromised. Another approach would involve gently modifying a monopolar channel, just enough to avoid unintentional facial nerve stimulation, The mathematical analysis estimates the strength of the current outflux near a recording electrode for a certain influx at a stimulation electrode. Suppose 8% of the current would flow out of the cochlear near EL10 when stimulating EL20, and that EL10 would be an unintentional facial nerve stimulation trigger. In this case, an approach could be to actively sink 8% of the current through EL10 when stimulating EL20. Instead of the current obeying Ohm's law and flowing out of the cochlear through the highly conductive path near EL10, EL10 would actively force 8% of the current to return through that electrode. This technique should avoid unintentional facial nerve stimulation while minimally interfering with the normal intracochlear field of monopolar stimulation at EL20. Thus, an algorithm can be designed that uses the TIM information to redefine the channels, turning them into quasi-monopolar channels (partial bipolar channels), in order to avoid unintentional facial nerve stimulation. FIG. 8B illustrates this case.

FIG. 8B is a graphical view illustrating an example where each monopolar field is modified to actively sink some current through an identified electrode (e.g., EL14 in FIG. 8B), where the actively sunk current percentage through the intracochlear electrode matches the passive conduction through the facial nerve canal, in connection with the post-op treatment algorithm (ALGO5) and method 800 of FIG. 8A. At EL14, a certain percentage of the current is actively sunk, driving the intracochlear voltage to zero. In doing so, the intracochlear current has no incentive to take the facial nerve canal route to leave the cochlea.

Thus, the post-op treatment algorithm (ALGO5) described above and in connection with method 800 of FIG. 8A can adjust spatial channel definition to avoid unintentional facial nerve stimulation (e.g., quasi-monopolar to avoid current flow along the facial nerve). This is potentially a stand-alone idea that can be independent of any objective measures from the other algorithms and methods described above. This automated solution focuses on novel multipolar methods that are specifically designed to keep the current away from the facial nerve canal in order to treat unintentional facial nerve stimulation.

In summary, the above disclosure in connection with FIGS. 3, 4, 5, 6, 7, 8A and 8B describes five algorithmic approaches (methods 300, 400, 600, 700, 800) to holistically manage unintentional non-auditory nerve stimulation (e.g., unintentional facial nerve stimulation), respectively:

(1) The first element (Pre-op ALGO1, method 300 of FIG. 3) is an unintentional facial nerve stimulation estimator for the pre-op scenario where a CT or MRI image is available. A segmentation algorithm traces the FACIAL NERVE canal and first cochlear turn, and then calculates the nearest geometrical distance (and possibly electrical distance by taking onto account the bone density) to basal turn. This information is presented to an expert surgeon for them to adapt electrode selection, surgical technique, and electrode position.

(2) The second element (Intra-op ALGO2, method 400 of FIG. 4) is a diagnostic based on the transimpedance matrix (TIM). While the surgeon and recipient are still inside the operating room, a TIM measurement is conducted, and an intelligent agent analyzes the TIM data for the occurrence of a third current sink. If a third current sink is found, its strength and extent can be determined. The purpose is to inform the surgeon to take surgical intervention (e.g., to reposition the electrode) in the OR.

(3) The third element (Post-op Diagnostic ALGO3, method 600 of FIG. 6) is a diagnostic during first fit, assisting the audiologist with the localization of the facial nerve (in terms of electrodes), and possibly informing the audiologist of the likely location of the most sensitive electrode/channel provoking unintentional facial nerve stimulation. Such diagnostic algorithm could be guided with prior knowledge from the operating room analysis (e.g., captured via Intra-op ALGO2), if available. This diagnostic tool can help the medical practitioner to deal with this complex case. Alternatively, a variation of ALGO1 could be used such that, at this point, with a post-op CT, which could be geometrically analyzed. In other words, although ALGO1 is described above as pre-op only (no stimulating assembly yet inserted), but is to be appreciated that ALGO1 could also or alternatively be used as part of an intra-op or even a post-op analysis.

(4) The fourth element (Post-op Recommendation ALGO4, method 700 of FIG. 7) builds further on the previous diagnostic tool (e.g., Diagnostic ALGO3) by also supporting treatment (map creation). An algorithm can recommend monopolar map optimizations (limit levels or deactivate).

(5) The fifth element (Post-op Treatment ALGO5, method 800 of FIG. 8A) is again a treatment recommendation algorithm. Building further on the previous idea, this recommendation algorithm suggests optimal changes to the spatial channel definition, with as objective to keep the current away of the FN. The new channel definitions depend on the capabilities of the chip set, an analysis of the intracochlear electrical fields, and can suggest changes from monopolar to bipolar or all-polar (common ground) in the case of more simple implantable receiver/stimulators, or be more advanced and personalized, based on precision analysis of TIM (e.g., on receiver/stimulators with multipolar capability).

It is to be appreciated that the above examples are merely illustrative and that the various techniques are not mutually exclusive. For example, in an example surgical use case, a combination of ALGO1 and ALGO2 could be used to achieve improved results. An ALGO1+ALGO2 approach would combine the anatomical knowledge (imaging) and electrical measurements (TIM) to make more accurate FNS risk assessments (Higher sensitivity+specificity). If both ALGO1+ALGO2 point in the direction of some FNS risk, then the two algorithms reinforce each other, or vice versa.

As noted above, certain aspects are described as being “pre-op,” “intra-op,” or “post-op.” These descriptions are merely for ease of illustration and do not limit any of the techniques (e.g., algorithms) to any specific timing, order, etc. That is, in certain examples, a technique that is described as being pre-op could be implemented as intra-op or post-op process; a technique that is described as being intro-op could be implemented as pre-op or post-op process; and a technique that is described as being post-op could be implemented as intra-op or pre-op process.

All the above approaches fit within the vision whereby in their simplest form, the algorithms inform the clinician providing a new piece of diagnostic information, nothing more. In this first stage, the clinician then decides on the further course of action (Human-in-the-loop). In a further stage, the algorithm not only provides the new diagnostic info, but also recommends also a certain stage of action, which the medical practitioner can accept or decline (Human-on-the-loop). This model is more powerful, as it can be used by a larger group of clinicians, as the need for expert knowledge is reduced. To reach this second stage, more clinical evidence can be required to support the recommendation claims. Finally, the third stage is the fully automated stage, where the unintentional facial nerve stimulation prediction/detection algorithm makes the decision to deactivate or reconfigure channels, without the involvement of a medical practitioner (human-out-of-the-loop). Such an automation scenario would be needed in a 100% self-care clinical model. For the foreseeable future, however, the management of complex cases such as unintentional facial nerve stimulation will usually be performed by medical practitioners (guided by the assistance provided by the unintentional facial nerve stimulation prediction/detection algorithms).

The techniques described above can provide one or more benefits and advantages over the state of the art, including but not limited to better hearing outcomes for the recipients impacted, reducing the need for reimplantation, lower complication rates for surgeons, enabling audiologists to perform their job quicker and reach better outcomes, and reducing the number of poor performers. Clinicians would benefit from some guidance during surgery and audiological programming based on objective measures. Also, considering that the cochlea can change over time with some chronic diseases (e.g., otosclerosis) such that a recipient can develop unintentional facial nerve stimulation some years after initial implantation, techniques that can help avoid or reduce the need for reimplantation have significant value.

As previously noted, for purposes of illustration, the techniques presented herein have primarily described with reference to cochlear implant systems and with reference to a specific type of unintentional stimulation or stimulation side effect, namely unintentional facial nerve stimulation. However, also as noted above, it would be appreciated that the techniques presented herein can also be partially or fully implemented by other types of implantable medical devices for management of different types of unintentional stimulation and/or stimulation side effects. That is, aspects of the techniques presented herein can be apply in other fields, such as with deep brain stimulators that are also prone to generate stimulation side effects, as well as neuromodulation applications where nearby nerves run through bony channels and stimulation can go too broad, such as spinal cord stimulators, a pelvic stimulators, stimulation devices to treat migraines, etc.

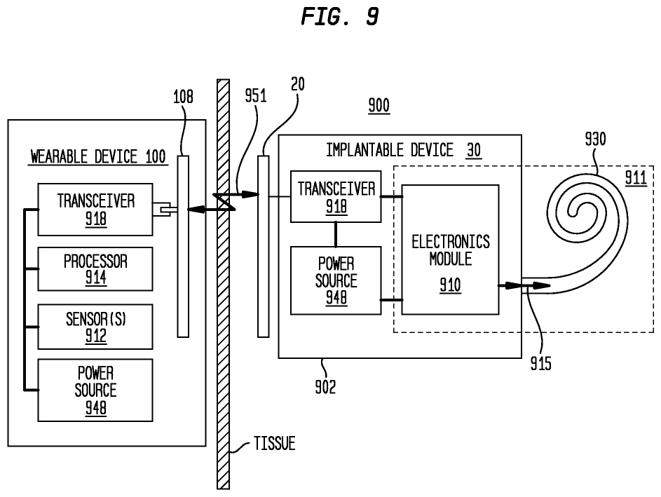

Several example devices that can benefit from technology disclosed herein are described in more detail in FIGS. 9-11, below. For example, the techniques described herein can be used to prioritize clinician tasks associated with configuring the operating parameters of wearable medical devices, such as an implantable stimulation system as described in FIG. 9, a vestibular stimulator as described in FIG. 10, or a retinal prosthesis as described in FIG. 11. As noted elsewhere herein, these devices are merely illustrative and that the techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, spinal cord stimulations, deep brain stimulators, pelvic stimulators, stimulation devices to treat migraines, as well as other medical devices that deliver stimulation to tissue.

FIG. 9 is a functional block diagram of an implantable stimulator system 900 that can benefit from the technologies described herein. The implantable stimulator system 900 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 902. Here, the wearable device 100 is configured to transcutaneously couple with the implantable device 30 via a wireless connection to provide additional functionality to the implantable device 30.

In the illustrated example, the wearable device 100 includes one or more sensors 912, a processor 914, a transceiver 918, and a power source 948. The one or more sensors 912 can be one or more units configured to produce data based on sensed activities. In an example where the stimulation system 900 is an auditory prosthesis system, the one or more sensors 912 include sound input sensors, such as a microphone, an electrical input for an FM hearing system, other components for receiving sound input, or combinations thereof. Where the stimulation system 900 is a visual prosthesis system, the one or more sensors 912 can include one or more cameras or other visual sensors. Where the stimulation system 900 is a cardiac stimulator, the one or more sensors 912 can include cardiac monitors. The processor 914 can be a component (e.g., a central processing unit) configured to control stimulation provided by the implantable device 30. The stimulation can be controlled based on data from the sensor 912, a stimulation schedule, or other data. Where the stimulation system 900 is an auditory prosthesis, the processor 914 can be configured to convert sound signals received from the sensor(s) 912 (e.g., acting as a sound input unit) into signals 951. The transceiver 918 is configured to send the signals 951 in the form of power signals, data signals, combinations thereof (e.g., by interleaving the signals), or other signals. The transceiver 918 can also be configured to receive power or data. Stimulation signals can be generated by the processor 914 and transmitted, using the transceiver 918, to the implantable device 30 for use in providing stimulation.

In the illustrated example, the implantable device 30 includes a transceiver 918, a power source 948, and a medical instrument 911 that includes an electronics module 910 and a stimulator assembly 930. The implantable device 30 further includes a hermetically sealed, biocompatible implantable housing 902 enclosing one or more of the components.

The electronics module 910 can include one or more other components to provide medical device functionality. In many examples, the electronics module 910 includes one or more components for receiving a signal and converting the signal into the stimulation signal 915. The electronics module 910 can further include a stimulator unit. The electronics module 910 can generate or control delivery of the stimulation signals 915 to the stimulator assembly 930. In examples, the electronics module 910 includes one or more processors (e.g., central processing units or microcontrollers) coupled to memory components (e.g., flash memory) storing instructions that when executed cause performance of an operation. In examples, the electronics module 910 generates and monitors parameters associated with generating and delivering the stimulus (e.g., output voltage, output current, or line impedance). In examples, the electronics module 910 generates a telemetry signal (e.g., a data signal) that includes telemetry data. The electronics module 910 can send the telemetry signal to the wearable device 100 or store the telemetry signal in memory for later use or retrieval.

The stimulator assembly 930 can be a component configured to provide stimulation to target tissue. In the illustrated example, the stimulator assembly 930 is an electrode assembly that includes an array of electrode contacts disposed on a lead. The lead can be disposed proximate tissue to be stimulated. Where the system 900 is a cochlear implant system, the stimulator assembly 930 can be inserted into the recipient's cochlea. The stimulator assembly 930 can be configured to deliver stimulation signals 915 (e.g., electrical stimulation signals) generated by the electronics module 910 to the cochlea to cause the recipient to experience a hearing percept. In other examples, the stimulator assembly 930 is a vibratory actuator disposed inside or outside of a housing of the implantable device 30 and configured to generate vibrations. The vibratory actuator receives the stimulation signals 915 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 918 can be components configured to transcutaneously receive and/or transmit a signal 951 (e.g., a power signal and/or a data signal). The transceiver 918 can be a collection of one or more components that form part of a transcutaneous energy or data transfer system to transfer the signal 951 between the wearable device 100 and the implantable device 30. Various types of signal transfer, such as electromagnetic, capacitive, and inductive transfer, can be used to usably receive or transmit the signal 951. The transceiver 918 can include or be electrically connected to a coil 20.

As illustrated, the wearable device 100 includes a coil 209 for transcutaneous transfer of signals with the concave coil 20. As noted above, the transcutaneous transfer of signals between coil 209 and the coil 20 can include the transfer of power and/or data from the coil 209 to the coil 20 and/or the transfer of data from coil 20 to the coil 209. The power source 948 can be one or more components configured to provide operational power to other components. The power source 948 can be or include one or more rechargeable batteries. Power for the batteries can be received from a source and stored in the battery. The power can then be distributed to the other components as needed for operation.

As should be appreciated, while particular components are described in conjunction with FIG. 9, technology disclosed herein can be applied in any of a variety of circumstances. The above discussion is not meant to suggest that the disclosed techniques are only suitable for implementation within systems akin to that illustrated in and described with respect to FIG. 9. In general, additional configurations can be used to practice the methods and systems herein and/or some aspects described can be excluded without departing from the methods and systems disclosed herein.

FIG. 10 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 2070. As such, the external device 1004 is configured to transfer data (and potentially power) to the vestibular stimulator 1012.

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 1034 also includes an internal/implantable coil 1014 that is generally external to the housing 1038, but which is connected to the transceiver via a hermetic feedthrough (not shown).

The stimulating assembly 1016 comprises a plurality of electrodes 2054(1)-(3) disposed in a carrier member (e.g., a flexible silicone body). In this specific example, the stimulating assembly 1016 comprises three (3) stimulation electrodes, referred to as stimulation electrodes 2054(1), 2054(2), and 2054(3). The stimulation electrodes 2054(1), 2054(2), and 2054(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 1012, the external device 1004, and/or another external device, can be configured to implement the techniques presented herein. That is, the vestibular stimulator 1012, possibly in combination with the external device 1004 and/or another external device, can include an evoked biological response analysis system, as described elsewhere herein.

FIG. 11 illustrates a retinal prosthesis system 2111 that comprises an external device 2111 (which can correspond to the wearable device 100) configured to communicate with a retinal prosthesis 2110 via signals 1151. The retinal prosthesis 2110 comprises an implanted processing module 2135 (e.g., which can correspond to the implantable device 30) and a retinal prosthesis sensor-stimulator 1190 is positioned proximate the retina of a recipient. The external device 2111 and the processing module 2135 can communicate via coils 209, 20.

In an example, sensory inputs (e.g., photons entering the eye) are absorbed by a microelectronic array of the sensor-stimulator 1190 that is hybridized to a glass piece 1192 including, for example, an embedded array of microwires. The glass can have a curved surface that conforms to the inner radius of the retina. The sensor-stimulator 1190 can include a microelectronic imaging device that can be made of thin silicon containing integrated circuitry that convert the incident photons to an electronic charge.

The processing module 2135 includes an image processor 2133 that is in signal communication with the sensor-stimulator 1190 via, for example, a lead 2198 which extends through surgical incision 2199 formed in the eye wall. In other examples, processing module 2135 is in wireless communication with the sensor-stimulator 1190. The image processor 2133 processes the input into the sensor-stimulator 1190, and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire and a signal is sent to the optic nerve, thus inducing a sight perception.

The processing module 2135 can be implanted in the recipient and function by communicating with the external device 2111, such as a behind-the-ear unit, a pair of eyeglasses, etc. The external device 2111 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
obtaining a pre-operative image of a recipient;
analyzing, with a computing device, the pre-operative image to determine a risk of unintentional stimulation associated with insertion of a stimulating assembly into the recipient; and
outputting, with the computing device, information relating to the risk of unintentional stimulation associated with insertion of the stimulating assembly into the recipient.

2. The method of claim 1, further comprising:
obtaining a post-operative image of the recipient; and
analyzing, with the computing device, the pre-operative image and the post-operative image to determine a risk of unintentional stimulation associated with insertion of the stimulating assembly into the recipient.

3. The method of claim 1, wherein obtaining the pre-operative image comprises:
obtaining a pre-operative magnetic resonance imaging (MRI) scan of an inner ear of the recipient.

4. The method of claim 1, wherein analyzing the pre-operative image to determine the risk of unintentional stimulation associated with insertion of the stimulating assembly into the recipient comprises:
analyzing the pre-operative image for presence of cochlear-facial dehiscence.

5. The method of claim 4, wherein outputting information relating to the risk of unintentional stimulation associated with insertion of the stimulating assembly into the recipient comprises:
outputting information characterizing the cochlear-facial dehiscence.

6. The method of claim 1, wherein analyzing the pre-operative image to determine the risk of unintentional stimulation associated with insertion of the stimulating assembly into the recipient comprises:
analyzing the pre-operative image with a segmentation process to determine a closest distance between a facial canal of the recipient and a basal turn of an inner ear of the recipient.

7. The method of claim 6, further comprising:
determining that the closest distance between the facial canal of the recipient and the basal turn of the inner ear is less than a threshold distance, and
wherein outputting information relating to the risk of unintentional stimulation associated with insertion of the stimulating assembly into the recipient comprises:
generating a warning output indicating that the recipient is at risk for unintentional facial nerve stimulation following insertion of the stimulating assembly into the inner ear.

8. The method of claim 7, wherein the risk for unintentional facial nerve stimulation is based, at least in part, on mechanical attributes of the stimulating assembly.

9. The method of claim 7, wherein the risk for unintentional facial nerve stimulation is based, at least in part, on a selected technique for insertion of the stimulating assembly into the inner ear.

10. The method of claim 7, wherein the warning output includes a probability of unintentional facial nerve stimulation and an estimate of a location within the inner ear, in terms of insertion angle, where the unintentional facial nerve stimulation is likely to occur.

11. The method of claim 4, wherein outputting information relating to the risk of unintentional stimulation associated with insertion of the stimulating assembly into the recipient comprises:
outputting a recommendation of a type of stimulating assembly to mitigate the risk of unintentional stimulation.

12. (canceled)

13. The method of claim 1, wherein the stimulating assembly is configured to be inserted into an inner ear of the recipient, and wherein the method further comprises:
following at least partial insertion of the stimulating assembly into the inner ear of the recipient;
performing a plurality of electrical measurements via electrodes of the stimulating assembly to obtain a plurality of electrical parameters for a position of the stimulating assembly within the inner ear; and
at the computing device, analyzing the plurality of electrical parameters to determine a risk of unintentional stimulation associated with the position of the stimulating assembly within the inner ear.

14. The method of claim 13, wherein performing a plurality of electrical measurements via the electrodes of the stimulating assembly comprises:
performing a plurality of voltage measurements to capture a plurality of voltages within the inner ear.

15. The method of claim 14, further comprising:
using the plurality of voltage measurements to determine at least one transimpedance matrix for the position of the stimulating assembly within the inner ear.

16. The method of claim 13, wherein analyzing the plurality of electrical parameters to determine the risk of unintentional stimulation associated with the position of the stimulating assembly within the inner ear comprises:
analyzing the plurality of electrical parameters to estimate one or more locations of current outflow from the inner ear at the position of the stimulating assembly within the inner ear.

17. The method of claim 13, further comprising:
outputting, with the computing device, information related to the risk of unintentional stimulation associated with the position of the stimulating assembly within the inner ear.

18. The method of claim 17, wherein the information related to the risk of unintentional stimulation comprises a location of a potential electrode that has an elevated risk of inducing unintentional stimulation from within the inner ear.

19. The method of claim 13, further comprising:
determining that the position of the stimulating assembly within the inner ear is associated with an elevated risk of unintentional stimulation; and
outputting a recommendation of a surgical intervention to reposition the stimulating assembly within the inner ear.

20. The method of claim 13, further comprising:
determining that the position of the stimulating assembly within the inner ear is associated with an elevated risk of unintentional stimulation; and
outputting a recommendation to insert a different type of stimulating assembly into the inner ear to mitigate the risk of unintentional stimulation.

21. The method of claim 13, wherein analyzing the plurality of electrical parameters to identify a risk of unintentional stimulation associated with the position of the stimulating assembly within the inner ear comprises:
analyzing the plurality of electrical parameters to estimate a risk of unintentional non-auditory stimulation associated with the position of the stimulating assembly within the inner ear.

22. The method of claim 13, wherein analyzing the plurality of electrical parameters to identify a risk of unintentional stimulation associated with the position of the stimulating assembly within the inner ear comprises:
analyzing the plurality of electrical parameters to estimate a risk of unintentional facial nerve stimulation associated with the position of the stimulating assembly within the inner ear.

23. The method of claim 13, wherein analyzing the plurality of electrical parameters to determine a risk of unintentional stimulation associated with the position of the stimulating assembly within the inner ear comprises:
determining, based on the plurality of electrical parameters, that the recipient has an elevated risk of unintentional facial nerve stimulation from at least one identified electrode of the stimulating assembly, wherein the stimulating assembly is a component of a medical device; and
setting at least one electrode channel configuration for use by the medical device in delivering electrical stimulation to the inner ear via the stimulating assembly to remediate the elevated risk of unintentional facial nerve stimulation from the at least one identified electrode.

24. The method of claim 23, wherein setting at least one electrode channel configuration for use by the medical device in delivering electrical stimulation to the inner ear via the stimulating assembly to remediate the elevated risk of unintentional facial nerve stimulation from at least one electrode comprises:
determining an amount of current outflow from the at least one electrode towards the facial nerve when electrical stimulation is delivered by the medical device via at least one other electrode of the stimulating assembly; and
configuring the medical device to, when delivering electrical stimulation via the at least one other electrode of the stimulating assembly, actively sink a first amount of current associated with the current outflow from the at least one identified electrode towards the facial nerve.

25. The method of claim 24, wherein the first amount of current comprises at least 50% of the current outflow from the at least one identified electrode towards the facial nerve.

26. (canceled)

27. (canceled)

28. (canceled)

29. (canceled)

30. One or more non-transitory computer readable storage media comprising instructions that, when executed by a processor, cause the processor to:
obtain a plurality of electrical values captured via electrodes of a stimulating assembly implanted in a recipient, wherein the stimulating assembly is a component of a medical device configured to deliver electrical stimulation signals to the recipient;
determine that the recipient has an elevated risk of stimulation side effects from at least one identified electrode of the stimulating assembly based on the plurality of electrical values; and
output a recommendation for setting at least one electrode channel configuration, for use by the medical device in delivering electrical stimulation to the recipient via the stimulating assembly, to remediate the elevated risk of stimulation side effects from the at least one identified electrode.

31. The one or more non-transitory computer readable storage media of claim 30, wherein the instructions executable to capture a plurality of electrical values via the electrodes of the stimulating assembly comprise instructions operable to:
obtain a plurality of voltage measurements representing a plurality of voltages within the recipient.

32. The one or more non-transitory computer readable storage media of claim 31, further comprising instructions executable to:
use the plurality of voltage measurements to determine at least one transimpedance matrix for the stimulating assembly within the recipient.

33. The one or more non-transitory computer readable storage media of claim 30, wherein the instructions executable to determine that the recipient has an elevated risk of stimulation side effects from at least one identified electrode of the stimulating assembly based on the plurality of electrical values comprise instructions executable to:
analyze the plurality of electrical values to estimate one or more locations of current outflow from the recipient.

34. The one or more non-transitory computer readable storage media of claim 30, further comprising instructions executable to:
estimate maximum stimulation levels on each channel of the stimulating assembly.

35. The one or more non-transitory computer readable storage media of claim 34, wherein the instructions executable to output a recommendation for setting at least one electrode channel configuration comprise instructions executable to:
output a recommendation to reduce the stimulation level of the at least one identified electrode below an upper limit to remediate the elevated risk of stimulation side effects from the at least one identified electrode.

36. The one or more non-transitory computer readable storage media of claim 34, wherein the instructions executable to output a recommendation for setting at least one electrode channel configuration comprise instructions executable to:
output a recommendation to deactivate the at least one identified electrode to remediate the elevated risk of stimulation side effects from the at least one identified electrode.

37. The one or more non-transitory computer readable storage media of claim 30, wherein the instructions executable to determine that the recipient has an elevated risk of stimulation side effects from at least one identified electrode of the stimulating assembly based on the plurality of electrical values comprise instructions executable to:
analyze the plurality of electrical values to estimate a risk of non-auditory stimulation from at least one identified electrode.

38. (canceled)

39. An apparatus, comprising:
an input device configured to obtain a plurality of electrical measurements captured via electrodes of a stimulating assembly positioned in an inner ear of a recipient; and
at least one processor configured to analyze the plurality of electrical measurements and to output an indication of a risk of unintentional non-auditory stimulation associated with the position of the stimulating assembly within the inner ear.

40. The apparatus of claim 39, wherein the plurality of electrical measurements comprise a plurality of voltage measurements.

41. (canceled)

42. The apparatus of claim 39, wherein the at least one processor is configured to analyze the plurality of electrical measurements to estimate one or more locations of current outflow from the inner ear at the position of the stimulating assembly within the inner ear.

43. The apparatus of claim 39 wherein the indication of a risk of unintentional non-auditory stimulation comprises a location of a potential channel that has an elevated risk of inducing unintentional non-auditory stimulation from within the inner ear.

44. The apparatus of claim 39, wherein the at least one processor is configured to output a recommendation of a surgical intervention to reposition the stimulating assembly within the inner ear or insert a different type of stimulating assembly into the inner ear to mitigate the risk of unintentional non-auditory stimulation.

45. (canceled)

46. (canceled)
